(19) United States
(12) Patent Application Publication
(10) Pub. No.: US 2013/0158403 A1
(43) Pub. Date: Jun. 20, 2013

(54) Method for Obtaining a Three-Dimensional Velocity Measurement of a Tissue

(71) Applicant: University Medical Devices, Inc., Ann Arbor, MI (US)

(72) Inventors: Paul G. Gottschalk, Ann Arbor, MI (US); James Hamilton, Brighton, MI (US)

(73) Assignee: University Medical Devices, Inc., Ann Arbor, MI (US)

(21) Appl. No.: 13/662,020

(22) Filed: Oct. 26, 2012

Related U.S. Application Data
(60) Provisional application No. 61/552,600, filed on Oct. 28, 2011.

(51) Int. Cl. A61B 8/06; A61B 8/08; A61B 8/00; A61B 8/14

(52) U.S. Cl. CPC A61B 8/065; A61B 8/145; A61B 8/486; A61B 8/483; A61B 8/466; A61B 8/5223; A61B 8/467; A61B 8/4444; A61B 8/54; A61B 8/58
USPC 600/447

(57) ABSTRACT

A method for obtaining a three-dimensional velocity measurement of a tissue from an ultrasound device comprising generating a set of correlation-velocity transfer functions from a first image plane and a second image plane, each image plane characterizing the tissue, wherein the set of correlation-velocity transfer functions can be applied to situations of constant ultrasound beam profile and/or periodic flow patterns; collecting, using the ultrasound device, an ultrasound measurement image; determining a set of in-plane velocity vectors and a set of speckle correlation values mapped to the ultrasound measurement image; determining a set of out-of-plane velocity vectors, corresponding to the set of in-plane velocity vectors, by applying the set of correlation-velocity transfer functions to the set of speckle correlation values; and generating, for the ultrasound measurement image, a three-dimensional velocity measurement from the sets of in-plane and out-of-plane velocity vectors.

### CROSS-REFERENCE TO RELATED APPLICATIONS

This application claims the benefit of U.S. Provisional Application No. 61/552,600 filed 28 Oct. 2011, titled “Method for Obtaining a Three-Dimensional Velocity Measurement of a Tissue”, which is incorporated in its entirety by this reference.

### TECHNICAL FIELD

This invention relates generally to the ultrasound field, and more specifically to an improved method for obtaining a three-dimensional velocity measurement of a fluid in the medical imaging field.

### BACKGROUND

Measurements of tissue properties can be used to assist in the assessment of health and functionality of organs and other distinct parts of an organism. For example, speckle tracking in ultrasound imagery may be used to measure tissue motion within a particular imaging plane, such as using ultrasound-based strain or strain rate images of heart muscle to measure the ability of the heart muscle to contract with high spatial and temporal resolution. As another example, quantifying speckle correlation may be used to measure out-of-plane tissue motion (e.g., fluid flow through a blood vessel), provided that it is possible to determine the transfer functions taking the degree of speckle correlation to the amount of out-of-plane tissue motion (tissue outside of a particular imaging plane). In conventional methods, these transfer functions are determined using a phantom, a mass mimicking the actual tissue to be measured. Typically the methods include (1) measuring the ultrasound beam profile of an ultrasound probe as a function of depth (of penetration) in a phantom and computing the transfer function based on various assumptions of the beam profile, or (2) empirically determining the transfer function directly, by moving the ultrasound beam probe at known distances over from the phantom and determining the degree of speckle correlation as a function of the ultrasound beam distance moved. In these conventional methods, the accuracy of the transfer function, and therefore accuracy of the measurement of out-of-plane tissue motion, is limited by the degree to which the phantom accurately mimics and is representative of the actual tissue, and by the correctness of any assumptions. The accuracy of the transfer function is further reduced when measuring tissue in motion, such as a moving fluid.

Thus, there is a need in the ultrasound field to create an improved method for obtaining a three-dimensional velocity measurement of a tissue. This invention provides such an improved method for obtaining a three-dimensional velocity measurement of a tissue.

### BRIEF DESCRIPTION OF THE FIGURES

FIG. 1 is a flowchart of the method for obtaining a three-dimensional velocity measurement of a tissue of a preferred embodiment;

FIG. 2 is a flowchart of information flow of the method for obtaining a three-dimensional velocity measurement of a tissue of a preferred embodiment;

FIG. 3 is a schematic of first and second image planes in the step of generating a set of correlation-velocity transfer functions in the method of a preferred embodiment;

FIG. 4 is a flowchart of the blocks of generating correlation-velocity transfer functions and applying correlation velocity transfer functions in the method of a preferred embodiment;

FIGS. 5A-5D are variations of the step of calculating, along a line of intersection of the first and second image planes, a set of correlation-velocity transfer functions in the method of a preferred embodiment; and

FIG. 6 is a schematic of a system and method for obtaining a three-dimensional velocity measurement of a tissue.

### DESCRIPTION OF THE EMBODIMENTS

The following description of embodiments of the invention is not intended to limit the invention to these preferred embodiments, but rather to enable any person skilled in the art to make and use this invention.

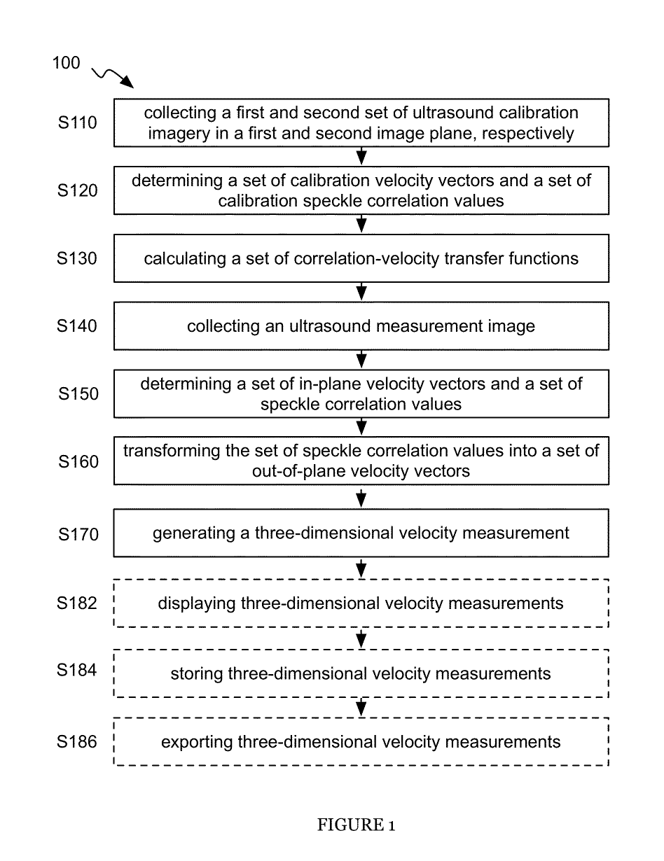

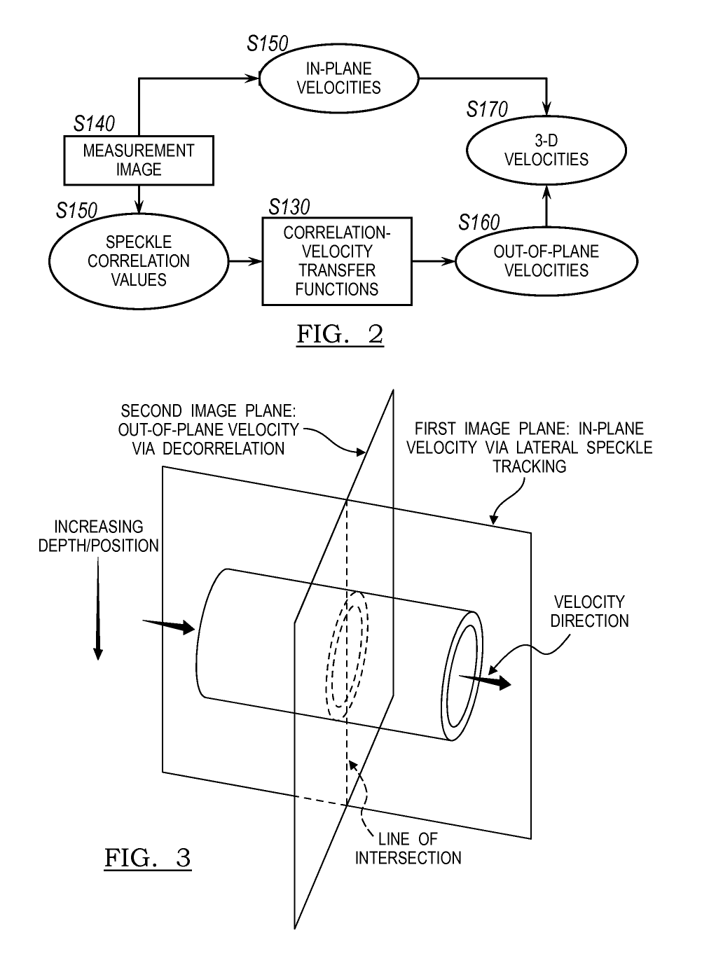

As shown in FIGS. 1 and 2, in a preferred embodiment, a method 100 for obtaining a three-dimensional velocity measurement of a tissue, wherein a portion of the tissue is characterized by a predominant axis that defines tissue motion, comprises the major steps of generating a set of correlation-velocity transfer functions S10 and applying the set of correlation-velocity transfer functions S20. Generating a set of correlation-velocity transfer functions S10 includes: collecting a first and second set of ultrasound calibration imagery in two image planes S110, determining a set of calibration velocity vectors of the tissue and a set of calibration speckle correlation values S120, and calculating a set of speckle correlation/out-of-plane velocity (also referred to as “correlation-velocity”) transfer functions S130 from the two image planes. Applying the set of correlation-velocity transfer functions S20 includes the steps of: collecting, with an ultrasound probe, an ultrasound measurement image S140; determining a set of in-plane velocity vectors and a set of speckle correlation values mapped to the ultrasound measurement image S150; determining a set of out-of-plane velocity vectors corresponding to the set of in-plane velocity vectors S160 by applying the set of correlation-velocity transfer functions to the set of speckle correlation values; and generating, for the ultrasound measurement image, a three-dimensional velocity measurement S170 from the in-plane and out-of-plane velocity vectors. The method is preferably used to measure blood flow patterns within a biological fluid vessel, such as vascular blood flow within a blood vessel or cardiac blood flow within heart chambers. Such assessment of blood flow patterns may be useful in applications such as clinical diagnostics and/or monitoring. However, the method may alternatively be used to characterize the movement or flow of any suitable biological tissue or other fluids in a fluid vessel.

### Generating Correlation-Velocity Transfer Functions

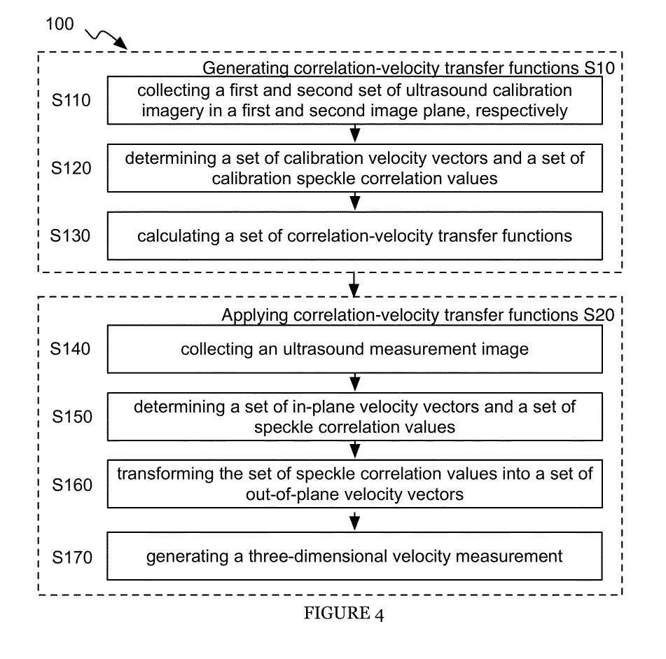

Generating a set of correlation/out-of-plane velocity transfer functions (or “correlation-velocity” transfer functions) S10, as shown in FIG. 4, comprises collecting a first set of ultrasound calibration imagery in a first image plane and second set of ultrasound calibration imagery in a second image plane S110, determining a set of calibration velocity vectors of the tissue and a set of calibration speckle correlation values S120, and calculating a set of speckle correlation-velocity transfer functions S130 that enable translation between speckle correlation values and out-of-plane velocity vectors derived within the first and second image planes. Generating a set of correlation-velocity transfer functions serves to generate a means for translating between measured speckle correlation values of an ultrasound planar image and motion outside of the plane of the image. Generating a set of correlation-velocity transfer functions preferably involves analyzing two calibration images taken in two substantially orthogonal imaging planes, as shown in FIG. 3, but can alternatively involve analyzing calibration images taken in non-orthogonal imaging planes, where geometric relationships between the non-orthogonal imaging planes are known. Each correlation-velocity transfer function preferably corresponds to a respective depth of signal penetration into tissue at positions along the line of intersection between the first and second image planes.

Collecting a first set of ultrasound calibration imagery in a first image plane and second set of ultrasound calibration imagery in a second image plane S110 comprises collecting a first set of ultrasound calibration imagery in a first image plane Sm. Collecting a first set of ultrasound calibration imagery S111 functions to gather ultrasound data for calibration purposes. The first image plane is preferably coincident with and substantially parallel to the axis of tissue motion, but alternatively, the first image plane may be coincident with and/or substantially parallel to a predominant axis of motion of another biological tissue. As shown in FIG. 3, in an example embodiment, the first image plane is coincident with and substantially parallel to the axis of blood flow within a blood vessel. The calibration images in this first plane are preferably collected prior to collecting the measurement images, although the calibration images in this first plane may additionally be used for measurement or other characterization of the flow velocity. Collecting a first set of ultrasound calibration imagery in a first image plane S111 includes positioning an ultrasound probe proximate to tissue, collecting raw ultrasound data from a plane coincident with the axis of tissue motion, and processing the raw ultrasound data to convert the raw ultrasound data into a suitable visual form, such as brightness mode (B-mode) images in the preferred embodiment. In alternative embodiments, the raw ultrasound data can be converted into another suitable visual form, such as A-mode, C-mode or M-mode forms. A sequence of ultrasound pulses is emitted and received over a period of time, and may be collected using any suitable steps. For example, pulses may be emitted and received in a manner known by one ordinarily skilled in the art, or similar to those described in U.S. Publication No. 2008/0021319 entitled “Method of modifying data acquisition parameters of an ultrasound device” and/or U.S. Publication No. 2010/0185093 entitled “System and method for processing a real-time ultrasound signal within a time window”, which are each incorporated in their entirety by this reference. However, the ultrasound imagery may be collected in any suitable manner and with any suitable ultrasound probe.

Collecting a first set of ultrasound calibration imagery in a first image plane and second set of ultrasound calibration imagery in a second image plane S110 also comprises collecting a second set of ultrasound calibration imagery in a second image plane S112. Collecting a second set of ultrasound calibration imagery in a second image plane S112 functions to gather additional ultrasound data for calibration purposes. In a preferred embodiment, the method includes rotating the ultrasound probe substantially 90 degrees from the first image plane used in step S111, such that the second, rotated image plane is substantially orthogonal to the first image plane. In an alternative embodiment, the second image plane is non-orthogonal to the first image plane, but a geometric relationship between the first image plane and the second image plane is known. As shown in FIG. 3, the second image plane in an example embodiment is transverse to the blood flow direction of blood in a blood vessel, such that blood in the blood vessel passes through the second image plane; in other words, the second image plane is preferably coincident with a lateral cross-section of the vessel. Collecting a second set of ultrasound calibration imagery S130 is preferably similar to the step of collecting a first set of ultrasound calibration imagery S120, except the second set of images is along a second image plane that intersects the first image plane.

Determining a set of calibration velocity vectors of the tissue and a set of calibration speckle correlation values S120 comprises determining a set of calibration velocity vectors S121 characterizing tissue motion within the first image plane. Determining a set of calibration velocity vectors S121 functions to gather, from the first set of calibration ultrasound data, in-plane velocity information used to generate the correlation-velocity transfer functions. Determining in-plane velocity vectors of the fluid within the first image plane preferably includes applying a speckle-tracking algorithm and obtaining lateral, or in-plane, velocities of tissue motion characterized within the first image plane. In the preferred embodiment, the speckle-tracking algorithm is applied to ultrasound data characterizing blood moving within the blood vessel in the first image plane, but in alternative embodiments the speckle-tracking algorithm is applied to ultrasound data characterizing motion of another tissue captured within the first image plane. Speckle tracking is a motion tracking method implemented by tracking the position of a kernel (section) of ultrasound speckles that are a result of ultrasound interference and reflections from scanned objects. The pattern of ultrasound speckles is substantially similar over small motions, which allows for tracking the motion of the speckle kernel within a search window (or region) over time. The speckle-tracking algorithm is preferably similar to that described in U.S. Publication Nos. 2008/0021319 and 2010/0185093, which are each incorporated in their entirety by this reference, and may include various algorithms such as normalized cross-correlation, but may alternatively be any suitable speckle-tracking algorithm. In alternative embodiments, determining velocity vectors of the tissue within the first image plane S122 may be performed using any suitable step(s) that obtain(s) in-plane, lateral velocities from the first set of ultrasound calibration imagery and/or raw ultrasound data.

Determining a set of calibration velocity vectors of the tissue and a set of calibration speckle correlation values S120 also comprises determining a set of calibration speckle correlation values of the second set of ultrasound calibration imagery S122. Determining a set of calibration speckle correlation values of the second set of ultrasound calibration imagery 122 functions to gather, from the ultrasound data, additional information used to generate the correlation-velocity transfer functions. The speckle correlation values are preferably values of a speckle correlation map derived from a normalized cross-correlation function obtained (e.g., as a byproduct) in applying a speckle-tracking algorithm to the second set of calibration imagery. The speckle-tracking algorithm is preferably similar to that used in determining velocity vectors characterizing tissue motion within the first image plane S122, but may alternatively be any suitable algorithm that obtains speckle correlation values. The steps of collecting a second set of calibration imagery S112 and determining a set of calibration speckle correlation values S122 collectively measure motion of the tissue in the second, rotated image plane at a line formed by the intersection between the first and second image planes. Along the common line formed by the intersection of the first and second image planes, the in-plane velocity vectors in the first image plane are the same vectors as the velocity vectors perpendicular to the second image plane.

Calculating, along a line of intersection of the first and second image planes, a set of correlation-velocity transfer functions S130 functions to generate accurate transfer functions based on the actual measured tissue of interest. Each of the correlation-velocity transfer functions preferably corresponds to a respective position along the line of intersection between the first and second image planes, or a respective depth. The number of data points, or potential number of correlation-velocity transfer functions, preferably scales with the resolution of ultrasound data; however, only a portion of the collected and processed data may be used in calculating a correlation-velocity transfer function. At each of these positions along the line of intersection is a data point corresponding to a calibration speckle correlation value/calibration velocity vector pair that may be used to generate a correlation-velocity transfer function. The calibration velocity vectors are determined from the speckle-tracking algorithm performed in step S122 (corresponding to the first image plane coincident a direction of tissue motion), and the calibration correlation values are determined from the speckle correlation map obtained in step S121 (corresponding to the second image plane orthogonal to the first image plane).

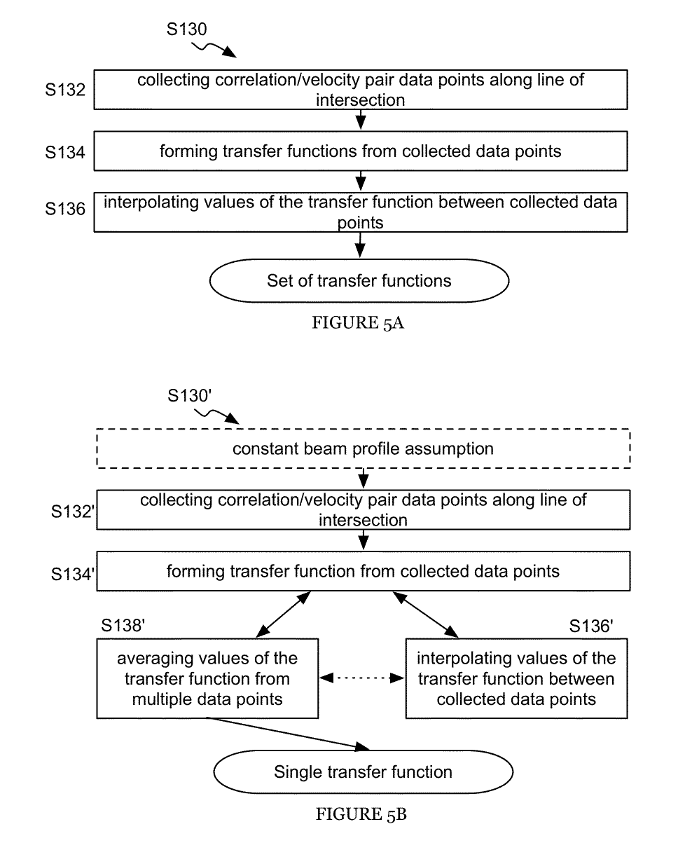

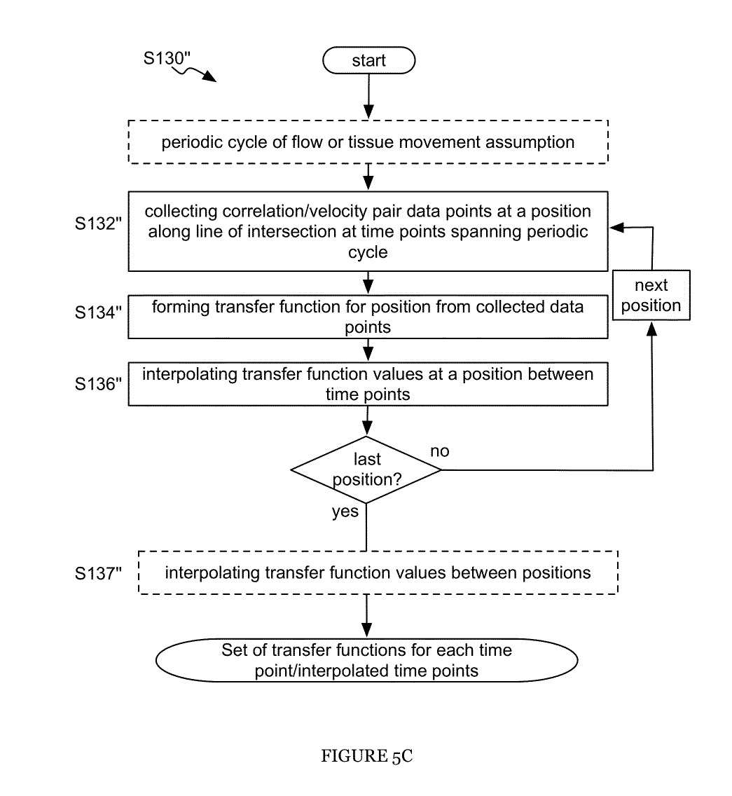

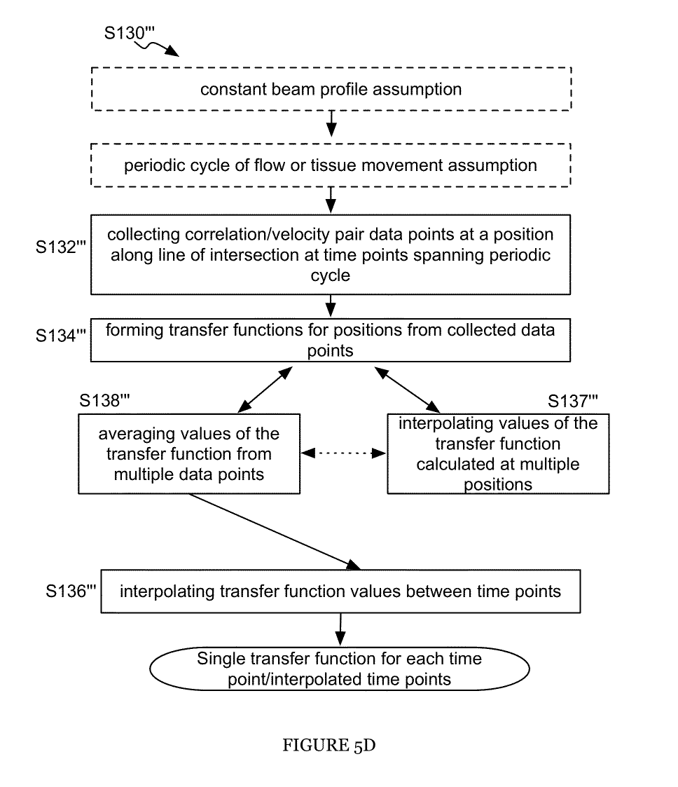

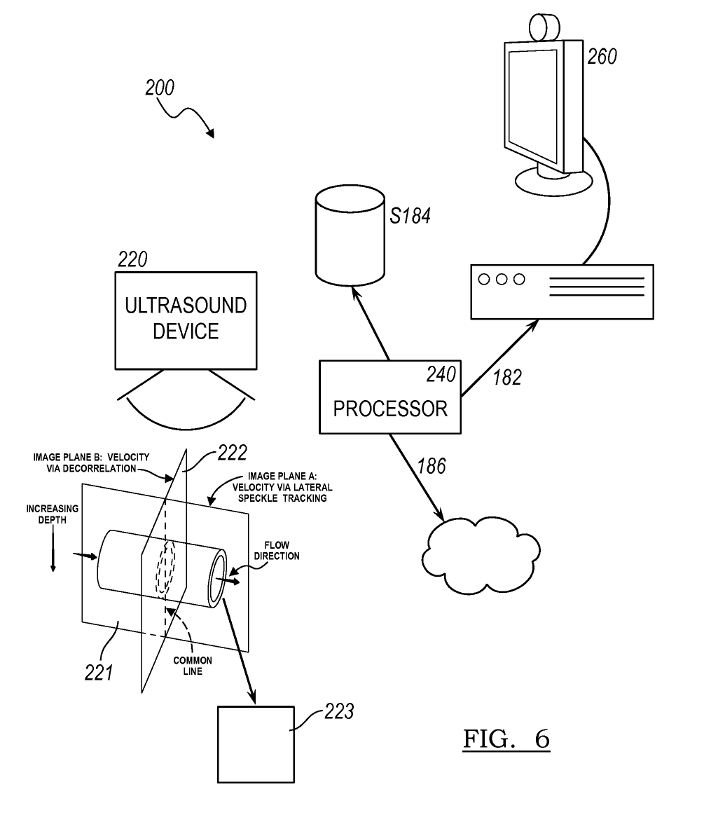

A first variation of calculating a set of correlation-velocity transfer functions S130, as shown in FIG. 5A, includes collecting the calibration speckle correlation value/calibration velocity vector pairs (i.e. data points) at positions along the common line of intersection S132, forming a correlation-velocity transfer function corresponding to each of the collected data points S134, and interpolating between values of the transfer functions corresponding to different data points S136. The interpolation may include any suitable interpolation method. Calculating a set of correlation-velocity transfer functions S130 may include additional variations.

A second variation of calculating a set of correlation-velocity transfer functions S130′, as shown in FIG. 5B, includes assuming that the ultrasound beam profile is substantially constant along the depth or extent of the tissue at positions along the line of intersection. For example, this assumption may be appropriate in applications in which the tissue of interest is of has a relatively small defining dimension (e.g. peripheral blood vessels). As a result of assuming a constant beam profile, all of the transfer functions are assumed to be substantially identical regardless of depth or position along the line of intersection of the first image plane and the second image plane. Therefore, a single frame of ultrasound data has a single correlation-velocity transfer function, and each position along the line of intersection contributes a data point to the same single correlation-velocity transfer function. In this second variation, calculating a set of correlation-velocity transfer functions may include collecting the calibration speckle correlation value/calibration velocity vector pairs (i.e. data points) at positions along the common line of intersection in a velocity frame S132′, forming a transfer function from the collected data points S134′, each corresponding to a position, and averaging values of the transfer function derived from multiple data points S138′ to obtain the single correlation-velocity transfer function. The averaging may include any suitable averaging method. Although the transfer function is assumed to be the same regardless of position along the line of intersection the steps of collecting, forming, and averaging may be accompanied by a step of interpolating between positions of the set of positions S136′ to generate additional correlation-velocity transfer functions corresponding to positions not of the set of positions. These additional correlation-velocity transfer functions may be averaged or otherwise combined with the correlation-velocity transfer functions corresponding to positions of the set of positions to generate a single transfer function characterizing the tissue along the line of intersection. Through this second variation of step S130 that assumes a substantially constant ultrasound beam profile, the set of correlation-velocity transfer functions includes a single correlation-velocity transfer function corresponding to all positions along the common line of intersection between the first and second image planes.

A third variation of calculating a set of correlation-velocity transfer functions S130″, as shown in FIG. 5C, includes assuming that motion of the tissue follows a periodic cycle (e.g., periodic blood flow patterns across cardiac cycles). In this variation, unlike in the first variation, the assumption that the ultrasound beam profile is substantially constant along the depth or extent of the tissue is not required. In this variation, calculating a set of correlation-velocity transfer functions includes: collecting the calibration speckle correlation value/calibration velocity vector pairs (i.e. data points) at a position along the line of intersection at a set of time points spanning all or part of the periodic cycle (e.g., cardiac cycle) S132″, forming a transfer function for the position from the collected data points S134″, interpolating values of the transfer function between data points corresponding to different time points S136″, and repeating the collecting, forming, and interpolating steps for all desired positions along the common of intersection. Similar to the first variation, the interpolating step may include any suitable interpolation method. Through this second variation of step S130, the set of correlation-velocity transfer functions comprises a series of correlation-velocity transfer function sets, each set comprising multiple transfer functions, wherein each transfer function corresponds to a position along the common line of intersection between the first and second image planes, and wherein each set corresponds to a time point of the set of time points, or a time point interpolated between two time points of the set of time points. Alternatively, in the third variation, calculating a set of correlation-velocity transfer functions S130″ can further include interpolation between positions of the set of positions in addition to interpolation between time points of the set of time points S137″, in order to further increase the number of generated correlation-velocity transfer functions that can be applied to an ultrasound measurement image.

A fourth variation of calculating a set of correlation-velocity transfer functions S130′″, as shown in FIG. 5D, includes assuming that the ultrasound beam profile is substantially constant (as in the assumption of the second variation) and that that motion of the tissue follows a substantially periodic cycle (as in the assumption of the third variation). In this fourth variation, calculating a set of correlation-velocity transfer functions includes: collecting the calibration speckle correlation value/calibration velocity vector pairs (i.e. data points) at a position along the line of intersection at a set of time points spanning all or part of the periodic cycle (e.g., cardiac cycle) S132′″, forming a transfer function for the position from the collected data points S134′″, and averaging values of the transfer function between data points S138′″. Although the transfer function is assumed to be the same regardless of position along the line of intersection (as in the second variation), the steps of collecting, forming, and averaging may be accompanied by a step of interpolating between positions of the set of positions to generate additional correlation-velocity transfer functions corresponding to positions not of the set of positions S137′″. These additional correlation-velocity transfer functions may be averaged or otherwise combined with the correlation-velocity transfer functions corresponding to positions of the set of positions to generate a single transfer function characterizing the tissue along the line of intersection. The fourth variation of S130′″ may also comprise interpolating values of the transfer function between data points corresponding to different time points S136′″. Through this fourth variation of step S130 that assumes a substantially constant ultrasound beam profile, the set of correlation-velocity transfer functions includes a single correlation-velocity transfer function, for each time point or time points interpolated between time points of the set of time points, corresponding to all positions along the common line of intersection between the first and second image planes.

The third and fourth variations of calculating a set of correlation-velocity transfer functions S130′″ may each further comprise receiving a signal from the tissue, wherein the signal is used to characterize of the period of time. In a first example, an electrocardiogram signal, cardiac magnetic resonance signal, or other signal measuring cardiac activity can be used to characterize a period of time defining a cardiac cycle, such that the method 100 is used to determine a three-dimensional velocity measurement of blood flow in a blood vessel. In a second example, an electromyography signal can be used to characterize a period of time defining a period of muscle activity, such that the method 100 is used to determine a three-dimensional velocity measurement of muscle tissue. In other examples, alternative signals can be used to characterize periods of time defining alternative periods of tissue motion.

Alternative variations of calculating a set of correlation-velocity transfer functions S130 include any combination and permutation of the steps and sub-steps. For example, in any of the variations of calculating a set of correlation-velocity transfer functions S130, the step of interpolating values of each of the transfer functions between data points may be performed after the entire set of transfer functions is formed, or may be respectively performed after each transfer function is formed. Furthermore, in alternative embodiments, the step of calculating a set of correlation-velocity transfer functions may include any suitable steps. Additionally, alternative assumptions that appropriately justify interpolating and/or averaging correlation-velocity transfer functions corresponding to positions and/or time points may be implemented to calculate a set of correlation-velocity transfer functions.

### Applying the Correlation-Velocity Transfer Functions

Applying the correlation-velocity transfer functions S20 comprises: collecting, with an ultrasound probe, an ultrasound measurement image S140; determining a set of in-plane velocity vectors and a set of speckle correlation values mapped to the ultrasound measurement image S150; determining a set of out-of-plane velocity vectors corresponding to the set of in-plane velocity vectors S160 by applying the set of correlation-velocity transfer functions to the set of speckle correlation values; and generating, for the ultrasound measurement image, a three-dimensional velocity measurement S170 from the in-plane and out-of-plane velocity vectors.

Collecting, with an ultrasound probe, an ultrasound measurement image S140 provides measureable data that is used to determine a set of in-plane velocity vectors and a set of speckle correlation values. Collecting an ultrasound measurement image S140 is preferably performed after calibration and calculating a set of correlation-velocity transfer functions S130. Collecting an ultrasound measurement image S140 may be similar to collecting a first set of ultrasound calibration imagery in a first plane and second set of ultrasound calibration imagery in a second plane S110, except that the measurement image may be obtained from any desired orientation relative to the tissue. Alternatively, collecting an ultrasound measurement image S140 may also further comprise collecting a set of ultrasound measurement images. Preferably, the ultrasound measurement image(s) is/are collected using the same ultrasound probe and other equipment used to collect the first and second sets of ultrasound calibration imagery, but the measurement image(s) and calibration imagery may alternatively be collected with different ultrasound equipment possessing sufficiently similar imaging characteristics. In some embodiments, the first and/or second sets of calibration imagery may be reused with the ultrasound measurement image(s) for measurement purposes. Alternatively, the ultrasound measurement imager(s) may be acquired in any other suitable manner (e.g., from storage).

Determining a set of in-plane velocity vectors and a set of speckle correlation values mapped to the ultrasound measurement image S150 functions to analyze the gathered ultrasound measurement data from the measurement imagery for flow velocity information. Determining a set of in-plane velocity vectors and a set of speckle correlation values S150 preferably includes applying a speckle-tracking algorithm to the collected ultrasound measurement image(s). The speckle-tracking algorithm is preferably similar to the one used in generating a set of correlation velocity transfer functions, in particular in determining a set of calibration velocity vectors of the tissue 121 and a set of calibration speckle correlation values S122. However, the speckle-tracking algorithm may be any suitable algorithm with sufficiently similar behavior to determine in-plane velocity vectors and speckle correlation value maps from the collected ultrasound measurement imagery. The method may further include analyzing the ultrasound measurement data to obtain any suitable information characterizing the tissue of interest.

Determining a set of out-of-plane velocity vectors corresponding to the set of in-plane velocity vectors S160 functions to convert the speckle correlation values from the ultrasound measurement image(s) into out-of-plane velocity vectors. Determining a set of out-of plane velocity vectors S160 preferably includes applying the set of correlation-velocity transfer functions calculated in step S130. As described above, each of the correlation-velocity transfer functions preferably corresponds to a respective position of the tissue of interest, and may also additionally correspond to a time point of a set of time points spanning a periodic cycle of tissue motion. The positions and/or time points represented by the ultrasound measurement image(s) may not exactly correspond to positions of the set of positions and/or time points of the set of time points, thus calling for use of correlation-velocity transfer functions calculated using averaging or interpolation Accordingly, at each position and/or time point in each speckle correlation map derived from the collected ultrasound measurement image(s), a correlation-velocity transfer function corresponding to that position and/or time point is preferably applied to convert the set of speckle correlation values (determined in step S150 and determined directly or by averaging and/or interpolation) to a set of out-of-plane velocity vectors corresponding to the set of in-plane velocity vectors. After applying the set of correlation-velocity transfer functions to the set of speckle correlation values, both in-plane and out-of-plane velocity vectors of the tissue of interest are known.

The step of generating, for the ultrasound measurement image, a three-dimensional (3D) flow velocity measurement S170 from the in-plane and out-of-plane velocity vectors functions to combine the determined velocity vectors into a multi-dimensional flow velocity characterization of the tissue of interest. Preferably, the in-plane velocity vectors are paired with out-of plane velocity vectors based on position and/or time point, and combined into a resultant vector in a manner known to one of ordinary skill in the art. The 3D flow velocity measurement preferably includes 3D flow vectors at each position in each collected or acquired ultrasound measurement image, which may be collected or acquired at time points spanning a period of time characterizing tissue motion (e.g. a cardiac cycle). The combination of the in-plane velocity vectors determined in step S150 and the out-of-plane velocity vectors determined in step S160 in some manners will be known and understood to one ordinarily skilled in the art; however, the in-plane and out-of-plane velocity vectors may be combined any suitable manner.

The method 100 may further include displaying, storing, and/or exporting the 3D flow velocity vector measurements, S182, S184, and S186, respectively, as shown in FIG. 6. The measurements may be displayed on a monitor or other user interface, stored on locally on a portable drive and/or remotely such as on a server, and/or exported to one or more various program applications or to another medium (e.g., printing). In one example, the displayed, stored, and/or exported 3D flow velocity measurement images may cover the entire measured portion of the tissue of interest, or may cover only a segment of the measured portion of the tissue of interest. As another example, such segments of the measured portion of the tissue of interest may include enlarged or “zoomed-in” areas of detail. In another example the 3D flow velocity measurement images representing different time points spanning a periodic cycle may be displayed, stored, and/or exported as a video that depicts evolution of 3D flow velocity measurements over time in the tissue.

The method of the preferred embodiment and variations thereof can be embodied and/or implemented using at least in part a machine configured to receive a computer-readable medium storing computer-readable instructions. The instructions are preferably executed by computer-executable components preferably integrated with the system and one or more portions of the processor 140 and/or the controller 150. The computer-readable medium can be stored on any suitable computer-readable media such as RAMs, ROMs, flash memory, EEPROMs, optical devices (CD or DVD), hard drives, floppy drives, or any suitable device. The computer-executable component is preferably a general or application specific processor, but any suitable dedicated hardware or hardware/firmware combination device can alternatively or additionally execute the instructions.

The FIGURES illustrate the architecture, functionality and operation of possible implementations of systems, methods and computer program products according to preferred embodiments, example configurations, and variations thereof. In this regard, each block in the flowchart or block diagrams may represent a module, segment, or portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

### System for Determining a Three-Dimensional Velocity Measurement of a Tissue

As shown in FIG. 6, a system 200 for obtaining a three-dimensional velocity measurement of a tissue comprises an ultrasound device 220 configured to collect a first set of calibration imagery in a first image plane 221, a second set of calibration imagery in a second image plane 222, and an ultrasound measurement image 223; a processor 240 configured to: calculate a set of correlation-velocity transfer functions from a first image plane and a second image plane, each image plane characterizing the tissue, determine a set of in-plane velocity vectors and a set of speckle correlation values mapped to the ultrasound measurement image, determine a set of out-of-plane velocity vectors, corresponding to the set of in-plane velocity vectors, by applying the set of correlation-velocity transfer functions to the set of speckle correlation values, and generate, for the ultrasound measurement image, a three-dimensional velocity measurement from the sets of in-plane and out-of-plane velocity vectors; and an interface 260 configured to display the three-dimensional velocity measurement. The system 200 is preferably configured to perform the method 100, or a portion thereof, described above.

As a person skilled in the art will recognize from the previous detailed description and from the figures and claims, modifications and changes can be made to the preferred embodiments of the invention without departing from the scope of this invention defined in the following claims.

We claim:

1. A method for obtaining a three-dimensional velocity measurement of tissue from an ultrasound device comprising:
collecting a first set of ultrasound calibration imagery in a first image plane and a second set of ultrasound calibration imagery in a second image plane that intersects the first image plane;
determining a set of calibration velocity vectors of the tissue and a set of calibration speckle correlation values;
calculating, along a line of intersection of the first image plane and a second image plane, a set of correlation-velocity transfer functions;
collecting an ultrasound measurement image;
determining a set of in-plane velocity vectors and a set of speckle correlation values mapped to the ultrasound measurement image;
transforming the set of speckle correlation values into a set of out-of-plane velocity vectors based on the set of speckle correlation values; and
generating, for the ultrasound measurement image, a three-dimensional velocity measurement from the sets of in-plane and out-of-plane velocity vectors.

2. The method of claim 1, wherein the first image plane is substantially orthogonal to the second image plane.

3. The method of claim 2, wherein at least one of the first image plane and the second image plane is coincident with a predominant axis of tissue motion.

4. The method of claim 1, wherein collecting the first set of ultrasound calibration imagery further comprises converting the first set of ultrasound calibration imagery into brightness mode (B-mode) data.

5. The method of claim 1, wherein determining a set of calibration velocity vectors of the tissue and a set of calibration speckle correlation values comprises:
determining a set of calibration velocity vectors of the tissue within the first image plane from the first set of ultrasound calibration imagery; and
determining a set of calibration speckle correlation values from the second set of ultrasound calibration imagery.

6. The method of claim 5, wherein determining a set of calibration velocity vectors of the tissue comprises applying a speckle tracking algorithm to the first set of ultrasound calibration imagery.

7. The method of claim 5, wherein determining a set of calibration speckle correlation values comprises applying a speckle tracking algorithm to the second set of ultrasound calibration imagery.

8. The method of claim 7, wherein applying a speckle tracking algorithm comprises obtaining a normalized cross-correlation function to derive a speckle correlation map that includes the set of calibration speckle correlation values.

9. The method of claim 1, wherein calculating, along a line of intersection of the first image plane and a second image plane, a set of correlation-velocity transfer functions comprises:
forming a set of correlation-velocity value pairs, each pair corresponding to a position of a set of positions along the line of intersection, based on the set of calibration velocity vectors and the set of calibration speckle correlation values; and
generating a set of correlation-velocity transfer functions based on the set of velocity-correlation value pairs.

10. The method of claim 9, wherein the set of correlation-velocity transfer functions is a single correlation-velocity transfer function along the line of intersection.

11. The method of claim 10, wherein the single correlation-velocity transfer function is obtained by averaging correlation-velocity transfer functions derived from at least two positions of the set of positions.

12. The method of claim 9, further comprising generating an interpolated correlation-velocity transfer function corresponding to a position between a first position and a second position of the set of positions along the line of intersection.

13. The method of claim 12, wherein the first position and the second position are adjacent positions of the set of positions.

14. The method of claim 9, wherein at least one of collecting a first set of ultrasound calibration imagery in a first image plane and collecting a second set of ultrasound calibration imagery in a second image plane comprises collecting data at a set of time points spanning a period of time.

15. The method of claim 14, further comprising receiving a signal from the tissue, wherein the signal is used to characterize of the period of time.

16. The method of claim 14, wherein calculating, along a line of intersection of the first image plane and a second image plane, a set of correlation-velocity transfer functions further comprises calculating a series of correlation-velocity transfer function sets, each correlation-velocity transfer function set corresponding to a time point in the set of time points.

17. The method of claim 16, further comprising generating an interpolated correlation-velocity transfer function, at a position of the set of positions, corresponding to a time point between a first time point and a second time point of the set of time points.

18. The method of claim 17, further comprising generating an interpolated correlation-velocity transfer function corresponding to a time point, at a position between a first position and a second position of the set of positions.

19. The method of claim 18, wherein the time point is between a first time point and a second time point of the set of time points.

20. The method of claim 19, wherein the first time point and the second time points are adjacent time points of the set of time points.

21. The method of claim 16, wherein each correlation-velocity transfer function set in the series of correlation-velocity transfer functions sets is a single correlation-velocity transfer function corresponding to a time point in the set of time points.

22. The method of claim 21, wherein each single correlation-velocity transfer function corresponding to a time point in the set of time points is obtained by averaging correlation-velocity transfer functions derived from at least two positions of the set of positions.

23. The method of claim 1, wherein collecting an ultrasound measurement image comprises collecting an ultrasound measurement image corresponding to one of the first image plane and the second image plane.

24. The method of claim 1, wherein determining a set of in-plane velocity vectors and the set of speckle correlation values mapped to the ultrasound measurement image comprises applying a speckle-tracking algorithm to the ultrasound measurement image.

25. The method of claim 1, wherein generating, for the ultrasound measurement image, a three-dimensional velocity measurement comprises combining an in-plane velocity vector from the set of in-plane velocity vectors and a corresponding out-of-plane velocity vector from the set of out-of-plane velocity vectors, into a resultant velocity vector.

26. The method of claim 1, further comprising displaying the three-dimensional velocity measurement.

27. A method for obtaining a three-dimensional velocity measurement of a tissue from an ultrasound device comprising:
calculating a set of correlation-velocity transfer functions from a first image plane and a second image plane, each image plane characterizing the tissue;
collecting, using the ultrasound device, an ultrasound measurement image characterizing the tissue;
determining a set of in-plane velocity vectors and a set of speckle correlation values mapped to the ultrasound measurement image;
determining a set of out-of-plane velocity vectors, corresponding to the set of in-plane velocity vectors, by applying the set of correlation-velocity transfer functions to the set of speckle correlation values; and
generating, for the ultrasound measurement image, a three-dimensional velocity measurement from the sets of in-plane and out-of-plane velocity vectors.

28. The method of claim 27, wherein calculating a set of correlation-velocity transfer functions comprises:
collecting a first set of ultrasound calibration imagery of the tissue in a first image plane;
determining a set of calibration velocity vectors of the tissue within the first image plane from the first set of ultrasound calibration imagery;
collecting a second set of ultrasound calibration imagery in a second image plane;
determining a set of calibration speckle correlation values from the second set of ultrasound calibration imagery; and
calculating, along a line of intersection of the first image plane and a second image plane, a set of correlation-velocity transfer functions.

29. The method of claim 27, wherein the first image plane is substantially orthogonal to the second image plane.

30. The method of claim 29, wherein at least one of the first image plane and the second image plane is coincident with a predominant axis of tissue motion.

31. The method of claim 28, wherein at least one of determining a set of calibration velocity vectors of the tissue and determining a set of calibration speckle correlation values comprises applying a speckle tracking algorithm to one of the first and second sets of ultrasound calibration imagery.

32. The method of claim 28, wherein calculating, along a line of intersection of the first image plane and a second image plane, a set of correlation-velocity transfer functions comprises:
at each position of a set of positions along the line of intersection, relating a velocity vector from the set of calibration velocity vectors to a speckle correlation value from the set of calibration speckle correlation values, thereby forming a set of correlation-velocity value pairs, and
generating a set of correlation-velocity transfer functions based on the set of velocity-correlation value pairs.

33. The method of claim 32, further comprising generating an interpolated correlation-velocity transfer function, at a position of the set of positions, corresponding to a position between a first position and a second position of the set of positions.

34. The method of claim 1, wherein generating, for the ultrasound measurement image, a three-dimensional velocity measurement comprises combining an in-plane velocity vector from the set of in-plane velocity vectors and a corresponding out-of-plane velocity vector from the set of out-of-plane velocity vectors, into a resultant velocity vector.

35. The method of claim 1, further comprising displaying the three-dimensional velocity measurement.

36. A system for obtaining a three-dimensional velocity measurement of a tissue comprising:
an ultrasound device configured to collect a first set of calibration imagery in a first image plane, a second set of calibration imagery in a second image plane, and an ultrasound measurement image;
a processor configured to:
calculate a set of correlation-velocity transfer functions from a first image plane and a second image plane, each image plane characterizing the tissue,
determine a set of in-plane velocity vectors and a set of speckle correlation values mapped to the ultrasound measurement image,
determine a set of out-of-plane velocity vectors, corresponding to the set of in-plane velocity vectors, by applying the set of correlation-velocity transfer functions to the set of speckle correlation values, and
generate, for the ultrasound measurement image, a three-dimensional velocity measurement from the sets of in-plane and out-of-plane velocity vectors; and
an interface configured to display the three-dimensional velocity measurement.
